(19) United States
(12) Patent Application Publication
(10) Pub. No.: US 2009/0326500 A1
(43) Pub. Date: Dec. 31, 2009

(54) SYSTEM AND METHOD FOR THE DELIVERY OF A DIAPER RASH PRODUCT

(76) Inventor: Mark Cantor, Nepean (CA)

Correspondence Address: MBM Intellectual Property Law LLP, 270 ALBERT STREET, 14TH FLOOR, OTTAWA, ONTARIO, K1P 5G8, CA

(21) Appl. No.: 12/300,045

(22) PCT Filed: May 4, 2007
(86) PCT No.: PCT/CA2007/000738

§ 371 Date: Jul. 15, 2009

Related U.S. Application Data
(60) Provisional application No. 60/746,770, filed on May 8, 2006.

(51) Int. Cl. A61F 13/45

(52) U.S. Cl. 604/385.06

(57) ABSTRACT

A system for delivering a diaper rash product is provided, said system comprising a pocket containing a measured amount of diaper rash product, with a means to facilitate the dispensing of said product by a caregiver when changing an infant's diaper.

### CROSS REFERENCE TO RELATED APPLICATIONS

This application is a 35 USC § 371 National Stage application of PCT Application No. PCT/CA07/00738 filed May 4, 2007; which claims the benefit under 35 USC § 119(e) to U.S. Application Ser. No. 60/746,770 filed May 8, 2006, now abandoned. The disclosure of each of the prior applications is considered part of and is incorporated by reference in the disclosure of this application.

### FIELD OF THE INVENTION

The present invention relates to product delivery systems and methods, and, in particular, to a system and method for the delivery of a diaper rash product.

### BACKGROUND OF THE INVENTION

Most babies have relatively sensitive skins, particularly in the genital region, and one of the most common skin problems with infants relates to diaper rash, also known as diaper dermatitis. Studies have shown that diaper dermatitis is one of the most common skin disorders in infants. (see, for example, Shin H T “Diaper dermatitis that does not quit” Dermatol Ther. 2005 March-April; 18(2):124-35)

The primary contributing factors to the development of diaper rash have long been thought to be infant urine and feces (Scheinfeld N. Diaper dermatitis: a review and brief survey of eruptions of the diaper area, Am J Clin Dermatol. 2005; 6(5):273-81). For example, infants under two months of age can urinate up to 20 times per day. Thereafter, infants can urinate up to 8 times a day. In addition, infant defecation can typically occur one or more times a day.

It had been theorized that the breakdown of the urine to yield ammonia primarily contributed to the formation of diaper rash by increasing the alkalinity of the skin. More recent studies have concluded that the combination of urine and feces are major contributors to the development of diaper rash. Urine has been shown to increase the pH of the diaper environment by breaking down urea in the presence of fecal urease. This rise in pH increases the activities of fecal proteases and lipases, which can damage skin (see Berg R W, Buckingham K W, Stewart R L. Etiologic factors in diaper dermatitis: the role of urine Pediatr Dermatol. 1986 February; 3(2):102-6 and Buckingham K W, Berg R W. Etiologic factors in diaper dermatitis: the role of feces Pediatr Dermatol. 1986 February; 3(2):107-12).

The skin irritation caused by diaper rash may also predispose an infant to irritation and infection. The two most common types of infection are those associated with yeast and bacteria. The most common yeast infection is caused by Candida albicans. Meanwhile, the most common bacterial infection is caused by Staphylococcus aureus.

Treatment for diaper rash involves removing the source of irritation, reducing the immediate skin reaction, relieving the discomfort, and preventing secondary infection and other complications. Frequent diaper changes and keeping the area as dry and clean as possible normally prove helpful. Keeping the area clean and dry, however, has not proven to be fully effective in protecting sensitive and/or already irritated skin from the chemical irritation associated with the by-products of infant urine and feces (see Atherton D, Mills K. What can be done to keep babies' skin healthy? RCM Midwives. 2004 July; 7(7):288-90).

In response to the problems associated with diaper rash, a number of products have been made available. Examples are Vaseline™, Aveeno™ Diaper Rash Cream, Johnson and Johnson™, Sudocrem™ Diaper Rash Cream, Isles Paste™, Balmex™, Desitin™, Triple Paste™ and others. These products are generally marketed in tubes and jars.

The principle function of these products has been to act as a protectant such that any potentially or actually affected area of skin is shielded against any further direct contact with urine or feces. The general method of applying diaper rash product is as follows: the caregiver first applies the product to their fingers and then rubs the product onto the infant's skin during diaper changing. Naturally, the efficacy of the diaper rash product is increased when it is applied directly to the area of the infant's skin affected by the rash.

Many preparations of diaper rash product contain zinc oxide in concentrations of between approximately 10% and 40% by weight. Typically, the zinc oxide is formulated with an oily substance such as petroleum which itself can also serve as a moisture barrier. Zinc oxide is insoluble and so provides better protection from moisture than petroleum alone. Studies have suggested that zinc oxide may also reduce infection and speed healing (see, for example MacKay D, Miller A L. Nutritional support for wound healing. Altern Med Rev. 2003; 8: 359-377).

Most of the diaper rash products currently available are in the form of an ointment or a water-in-oil emulsion. The high viscosity associated with these products keeps the diaper rash product from being washed away by urine or feces. In many cases, the high viscosity is the result of the inclusion of zinc oxide. Thus, the typical ointment prevents urine or feces from coming into direct contact with the skin and, as such, the product acts as a barrier. Unfortunately, the properties that make these products effective skin barriers also make the products difficult to remove from the hands of the parent or caregiver, even when using soap and water. This is especially problematic when an infant requires diaper changes away from the home and the caregiver is unable to remove the diaper rash product from their hands once the diaper has been changed. This is especially inconvenient at restaurants and social gatherings where clean hands are desirable. Diaper rash products are also known to stain fabrics such as clothing, furniture or changing pads.

A further disadvantage with traditional methods of applying diaper rash product is that the entire container of product must be transported by the caregiver along with diapers and other diaper changing paraphernalia.

Canadian Patent No. 1,225,026 to Buckingham discloses methods for the treatment of diaper dermatitis through the application of a pharmaceutically acceptable lipase-inhibiting agent that reduces the action of lipase on the skin. In one embodiment, the lipase-inhibiting agent is applied to the top inner surface of a diaper. The disadvantage to this design is that it uses more of the diaper rash treatment agent than may be necessary and does not allow for targeted application of the diaper rash treatment agent.

Canadian Patent No. 2,515,443 to Gabbay discloses a disposable diaper wherein the inner layer of the diaper possesses a plurality of fibres coated in diaper rash product. The disadvantage to this design is that it uses more of the diaper rash product than may be necessary and does not allow for targeted application of the diaper rash product. A further disadvantage to this design is the tendency of the diaper rash product to be absorbed by the material of the diaper, resulting in less transfer to the skin of the infant during use.

U.S. Pat. No. 6,803,045 to Goldberg discloses a diaper rash product applicator with an elongated housing and an elliptically domed applicator portion. The disadvantage to this design is that it is bulky, cannot be incorporated into the diaper itself and requires the caregiver to transport the applicator separately.

U.S. patent application Ser. No. 10/626,069 (Pub. No. 20050079229) to Cawthon discloses a system for applying diaper rash treatment compositions to a selected skin treatment area involving the use of an atomizing spray dispenser to apply the composition as a mist or spray. The disadvantage of this design is that a substantial amount of atomized composition is required to create the necessary water barrier. Also, this design cannot be incorporated into a diaper and requires the caregiver to transport the atomizer separately.

U.S. patent application Ser. No. 10/305,779 (Pub. No. 20040102750) to Jameson discloses an absorbent body structure such as a disposable diaper incorporating solid deposits of skin wellness lotion with a composition such that the solid deposits have a phase-change temperature that is less than body temperature. A second set of liquid deposits is also provided to act as a barrier once the solid deposits have been exhausted. In one embodiment the skin wellness lotion incorporates diaper rash treatment ingredients. The disadvantage to this design is the tendency of the diaper rash product to be absorbed by the material of the diaper, resulting in less transfer to the skin of the infant during use. A further disadvantage of this design is that it does not permit targeted application of the diaper rash product to the affected area of skin at the time the diaper is applied, thus reducing the effectiveness of the lotion.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a system and method for the delivering of a diaper rash product.

In accordance with one aspect of the present invention, there is provided a system for delivery of a diaper rash product to an affected area of skin, said system comprising: one or more pockets formed of an upper layer and a lower layer, wherein said upper layer overlays said lower layer, and the peripheral edge of the upper layer is sealingly affixed to the lower layer to form a sealed enclosure; each of said one or more pockets containing diaper rash product; and a dispensing means associated with each of said one or more pockets for dispensing said diaper rash product, wherein said one or more pockets are attached to a surface of a disposable diaper.

In accordance with another aspect of the present invention, there is provided a method for delivering a diaper rash product to an area of skin, said method comprising the steps of: providing a sealed pocket containing said diaper rash product, said sealed pocket operatively associated with a dispensing means; wherein said pocket is attached to a surface of a disposable diaper; dispensing said diaper rash product through said dispensing means to provide dispensed diaper rash product that remains associated with said pocket; and applying said dispensed diaper rash product from said pocket to said area of skin.

In accordance with another aspect of the present invention, there is provided a system for delivery of a diaper rash product to an affected area of skin, said system comprising: one or more pockets formed of an upper layer and a lower layer, wherein said upper layer overlays said lower layer, and the peripheral edge of the upper layer is sealingly affixed to the lower layer to form a sealed enclosure; each of said one or more pockets containing a measured amount of diaper rash product; a dispensing means associated with each of said one or more pockets for dispensing said diaper rash product; and an applicator means attached to a surface of a disposable diaper and operatively associated with one or more of said one or more pockets, wherein said applicator means comprises a tube comprising an open end and a sealed end, said sealed end being attached to said lower layer of said pocket.

### BRIEF DESCRIPTION OF THE DRAWINGS

These and other features of the invention will become more apparent in the following detailed description in which reference is made to the appended drawings.

FIG. 1 is an isometric view of the pocket attached to a disposable diaper according to one embodiment of the present invention.

FIG. 2 is a top view of the pocket depicted in FIG. 1.

FIG. 3 is a top view of the pocket with a frangible seal according to an alternative embodiment of the present invention.

FIG. 4 is a top view of the pocket with a slit and strip according to an alternative embodiment of the present invention.

FIG. 5 is a cross sectional view of the pocket according to one embodiment of the present invention.

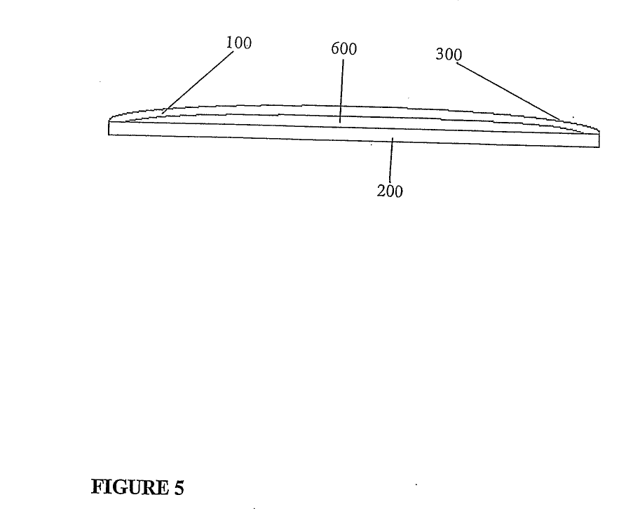

FIG. 6 is a top view of the pocket comprising a tube-like applicator means according to another embodiment of the present invention.

### DETAILED DESCRIPTION OF THE INVENTION

As used herein, the term “about” refers to a +/−10% variation from the nominal value. It is to be understood that such a variation is always included in any given value provided herein, whether or not it is specifically referred to.

Unless defined otherwise, all technical and scientific terms used herein have the same meaning as commonly understood by one of ordinary skill in the art to which this invention belongs

The present invention is directed towards providing a system and method for a caregiver to deliver a diaper rash product that can be distributed with or integrated into a disposable diaper. The system is designed so that contact between a caregiver's hands and the diaper rash product is minimized. The system comprises a sealed pocket containing a measured amount of diaper rash product and a dispensing means operatively associated with the pocket to allow said diaper rash product to be removed from the pocket. For convenience, the system is intended for distribution to the consumer together with a disposable diaper. According to various embodiments the system can, however, be distributed separately. In one embodiment, the pocket is designed so that it can be attached or, alternatively, integrated into a disposable diaper. According to one embodiment of the present invention, a dispensing means is used to breach the seal of the pocket, and the diaper can be grasped and used to apply the diaper rash product to the area of skin affected by the diaper rash, thus minimizing contact between the diaper rash product and the skin of a caregiver.

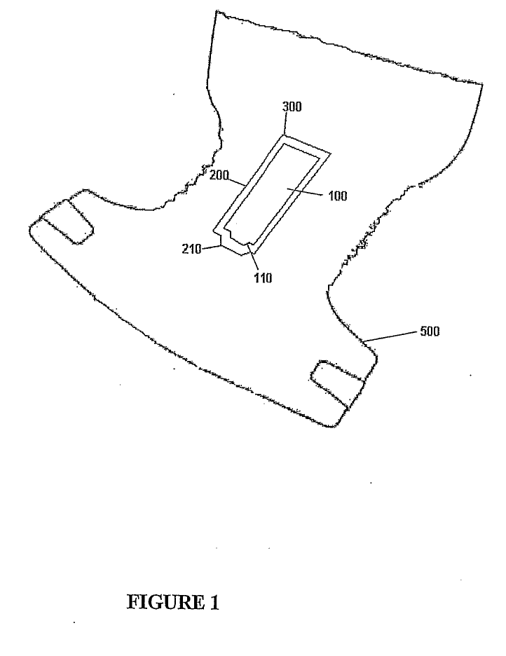

With reference to FIG. 1, according to an embodiment of the present invention, there is provided a pocket 300 which is attached to a disposable diaper 500. The details of disposable diaper construction are well known to a worker skilled in the art and the following description is provided for contextual purposes. See, for example Japanese Patent Application No. 1996-196565A, U.S. Pat. No. 3,688,767 to Goldstein, U.S. Pat. No. 4,834,738 to Kielpikowski, U.S. Pat. No. 4,904,251 to Takamitsu and others. The disposable diaper 500 comprises a front waist region 310, a back waist region 320 and a crotch region 330. The disposable diaper 500 and, in particular, the crotch region 330 comprises a laminate of a liquid-permeable top inner surface 340, a liquid-impermeable back surface (not shown) and a liquid-absorbent core disposed therebetween (not shown). In one embodiment of the present invention, the pocket 300 is attached to the top inner surface 340 of the crotch region 330. In alternative embodiments of the present invention, the pocket 300 can be located on the top inner surface 340 of the front waist region 310 or of the back waist region 320 so as to provide easy application to the buttocks or genital area of an infant depending on the infant and how the infant is positioned relative to the disposable diaper prior to changing. In an alternative embodiment of the present invention, multiple pockets 300 are provided on any of the front waist region 310, the crotch region 330 and/or the back waist region 320. In one embodiment of the present invention, the pocket 300 is placed on the diaper 500 so that, when the diaper 500 is worn by an infant, the pocket 300 is located away from the genital or anal areas of the infant so that it does not interfere with the diaper's 500 absorbent function.

### The Pocket

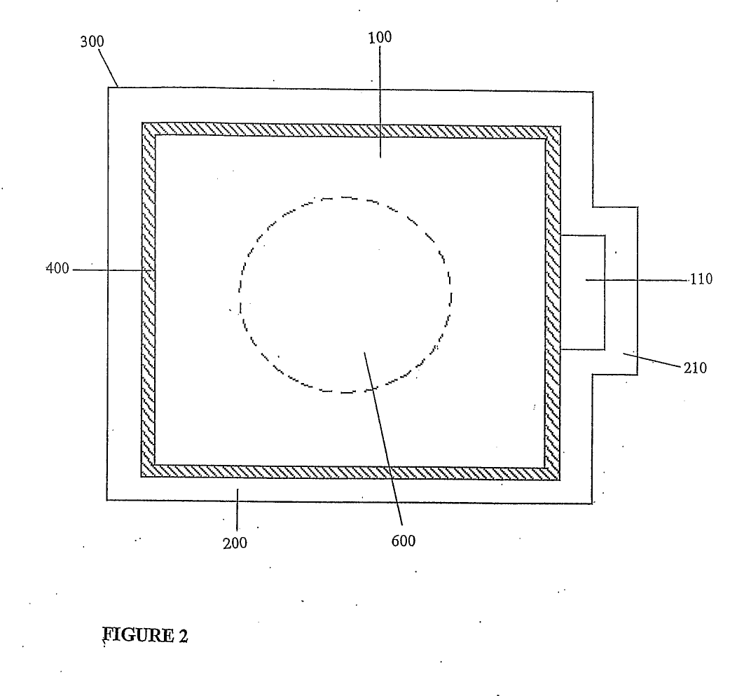

With reference to FIG. 2 which depicts one embodiment of the pocket component of the system according to the present invention, the pocket 300 is formed from an upper layer 100 and a lower layer 200 where the upper layer 100 overlies the lower layer 200. The upper layer 100 and lower layer 200 are formed of a thin, flexible impermeable material. A worker skilled in the art would appreciate that the material used to form the pocket 300 would need to be chemically inert, non-toxic, and capable of being sealed to form the pocket 300 and of sufficient tensile strength to contain the diaper rash product 600 during normal packaging and transport to consumer distributing centres and in a caregiving bag when outside of the home. Some non-limiting examples of suitable material are plastic (such as cellulose film, thermoplastic, low density polyethylene and the like), rubber (such as polyethylene, polyurethane, silicone rubber and the like), latex, waterproof paper or cloth, plasticized paper, foil or the like.

With reference to FIG. 2, the pocket 300 is formed by laying the upper layer 100 on top of the lower layer 200 and sealing the upper layer 100 to the lower layer 200 along the periphery of the upper layer 100 so as to form a seal 400 through which diaper rash product 600 cannot leak. A worker skilled in the art would recognize that the seal 400 can be created through various means, for example, heat-sealing, stitching, odourless, non-toxic adhesives or solvents or the like. The seal 400 must be sufficiently strong to contain the diaper rash product 600 during normal packaging and transport to consumer distributing centres and in a caregiving bag when outside of the home. In one embodiment of the present invention the upper 100 and lower layers 200 are removably sealed together with a weak adhesive that is strong enough to contain the diaper rash product 600 as noted above but is weak enough to allow the two layers 100 and 200 to be separated manually when desired, such as by peeling back the upper layer 100 of the pocket 300.

The pocket 300 is filled with a measured amount of diaper rash product 600 inserted between the upper layer 100 and lower layer 200 of the pocket prior to sealing.

Although the figures depict the pocket 300 as being generally rectangular in shape, a worker skilled in the art would also appreciate that alternate shapes are possible without affecting the function of the present invention. Non-limiting examples of alternative shapes for the pocket 300 are circles, ovals, or egg-shapes. Alternatively, if desired, the shape of the pocket could take an irregular shape such as a leaf or flower-like shape or could, for example, be shaped to mimic popular characters familiar to children.

### Optionally Attaching Pocket to Diaper

With reference to FIG. 1, in one embodiment, the pocket 300 is designed to be attached to a disposable diaper 500 at the time of manufacture. In one embodiment of the present invention, the lower layer 200 of the pocket 300 is attached to the top inner surface 340 of the diaper 500. A worker skilled in the art would appreciate that this attachment could be accomplished by various means, such as heat sealing, stitching or through the application of an odourless, non-toxic adhesive or solvent. In an alternative embodiment of the present invention, the top inner surface 340 of the diaper 500 comprises the lower layer 200 of the pocket 300. In this embodiment it may be necessary to treat the portion of the top inner surface 340 forming the lower layer 200 of the pocket 300 such that the diaper rash product will not be absorbed by the material of the top inner surface 340. In this embodiment, the pocket 300 should be located away from the genital or anal areas of the infant so that the less permeable section of the top inner surface 340 forming the lower layer 200 does not interfere with the diaper's 500 absorbent function when the diaper 500 is in use.

A worker skilled in the art would appreciate that the pocket 300 should be of sufficient size and located on the diaper 500 such that if pressure is applied to the diaper 500, for example, during packaging and transport, the integrity of the pocket 300 is not compromised. For example, while the size of the pocket will be dependant on the amount of diaper rash product contained therein, a worker skilled in the art would appreciate that a sufficiently large pocket 300, relative to the volume of diaper rash product, would allow the pocket 300 to endure some compression without the diaper rash product 600 escaping. A worker skilled in the art would also appreciate that a location on the diaper 500 that would subject the pocket 300 to a minimum of folding and pressure would be preferable.

### Dispensing Means

In order to remove the diaper rash product from the pocket so that it can be applied to an infant, the pocket is operatively associated with a dispensing means. In one embodiment, the dispensing means forms an integral part of the pocket. With reference to FIGS. 1 and 2, in one embodiment of the present invention, the dispensing means comprises a pull tab 110, formed from, or as an extension of, the upper layer 100 and which extends past the seal 400 between the upper layer 100 and the lower layer 200. The pull tab 110 can be grasped and used to pull the upper layer 100 away from the lower layer 200 to break the seal 400 of the pocket 300 and reveal the diaper rash product 600 contained therein. In this embodiment a weak adhesive would be necessary to allow the lower layer 200 and upper layer 100 to be physically separated when desired.

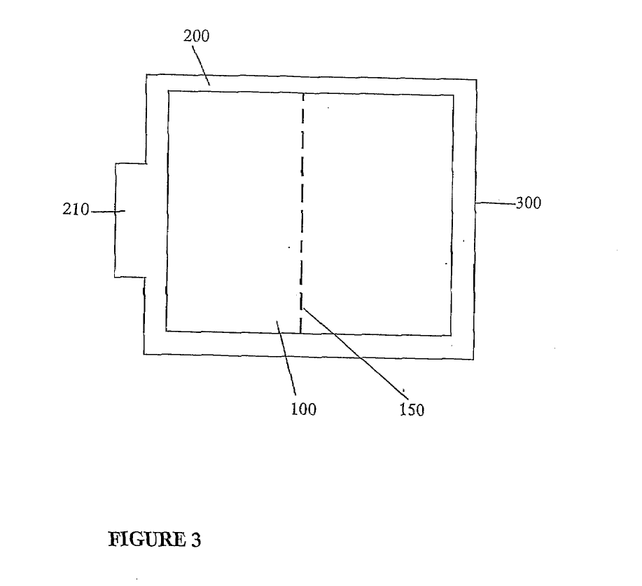

With reference to FIG. 3, according to an alternate embodiment of the present invention, the dispensing means comprises a frangible seal 150 in the material forming the upper layer 100. The frangible seal 150 extends substantially across the width of upper layer 100 and is comprised of a recess or score in the outer surface of the upper layer 100. A worker skilled in the art would appreciate that the frangible seal 150 should be sufficiently strong that it can contain the diaper rash product through the normal stresses of manufacture, packaging and transport but be weak enough to be easily broken when desired through the application of sufficient manual pressure to the upper layer 100 on either side of the frangible seal 150. The present invention also contemplates that the frangible seal 150 can extend substantially along the length or width of the upper layer 100, diagonally across the upper layer 100 or may extend along just a fraction of the respective dimension of the upper layer 100 provided that, once the seal is broken, the resultant opening is of a sufficient size to allow the diaper rash product to exit the pocket 300.

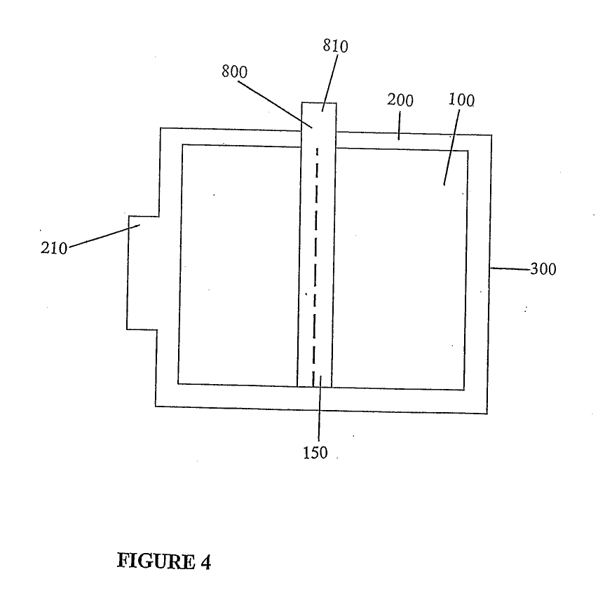

With reference to FIG. 4, according to an alternative embodiment of the invention, the dispensing means comprises a slit 130 in the material of the upper layer 100. The slit 130 extends substantially across the width of the upper layer 100. The present invention also contemplates that the slit 130 can extend substantially along the length of the upper layer 100, diagonally across the upper layer 100 or may extend along just a fraction of the respective dimension of the upper layer 100 provided that the slit 130 is of a sufficient size to allow the diaper rash product to exit the pocket 300. A strip 800 is removably attached to upper layer 100 by means of an odourless, non-toxic adhesive, covering the slit 130, such that the strip 800 seals the edges of the slit 130 together. The strip 800 is comprised of thin, flexible material such as plastic, paper, plasticized paper, latex or rubber. A worker skilled in the art would appreciate that the seal between the strip 800 and the edges of the slit 130 should be sufficient to contain the diaper rash product but weak enough to allow the strip 800 to be removed during use. Strip 800 further comprises a pull tab 810 that is not attached to either the upper layer 100 or lower layer 200. The tab 810 can be grasped so as to remove the strip 800 from the upper layer 100. Once the strip 800 is removed, the edges of the slit 130 are no longer joined and the diaper rash product is exposed for application.

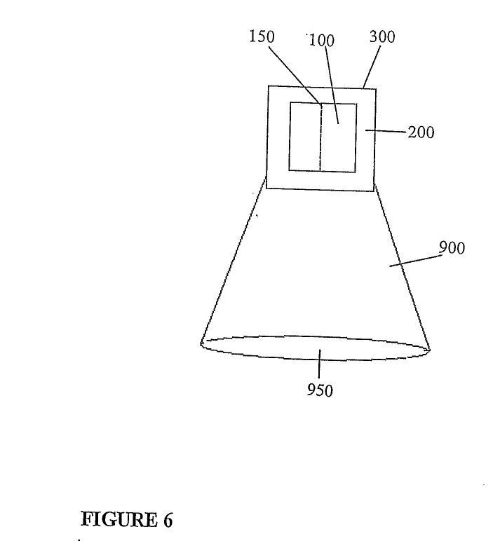

In a further embodiment of the invention, the pocket can further comprise an applicator means. With reference to FIG. 6, which illustrates an exemplary embodiment of an applicator means, pocket 300 comprises an applicator means that comprises a tube or tube-like structure 900 attached to the lower layer 200. The tube 900 is made of a non-toxic, flexible material for example cloth, plastic latex, rubber and the like. According to an alternative embodiment of the present invention, the tube 900 is formed in part from the material of lower layer 200. The tube comprises an end 910 which is farthest from the pocket 300 and an end 920, which is closest to the pocket 300. The end 920 of the tube 900 which is closest to the pocket 300 is sealed closed by means of heat sealing, a non-toxic adhesive, or the like. The tube 900 is of sufficient width to accommodate one or more adult human fingers. Once the diaper rash product (not shown) is removed via any of the various dispensing means discussed above, said one or more fingers can be placed into the tube 900 through the end 910 farthest from the pocket and used to move the pocket 300 so as to apply the diaper rash product in a controlled fashion. The fingers in the tube 900 apply pressure to the underside of the pocket 200 which forces the diaper rash product from the pocket 300 and onto the skin of the infant when in use. In one embodiment of the present invention, pressure between the fingers in the tube 900 and the skin of an infant can be used to breach a disposal means comprising a frangible seal.

In one embodiment of the present invention, the applicator means is attached to the inner surface of a diaper (not shown) such that the applicator means underlies the pocket 300 when attached. In an alternative embodiment of the present invention, the applicator means is integrated into the top inner surface of the diaper. In another alternative embodiment of the present invention, the pocket 300 and applicator means are provided separately from the diaper. In yet another embodiment of the present invention, the applicator means is removably attached to the top inner surface of the diaper by a means allowing for the applicator means to be easily manually separated from the diaper. A worker skilled in the art would appreciate that the attachment means should allow the applicator means to be separated from the diaper while preserving the integrity of both the top inner surface of the diaper and the applicator means. Another important consideration that a worker skilled in the art would appreciate is be to ensure that the removal does not result in any residue or uneven surfaces on the top inner surface of the diaper that would irritate the skin of an infant. Examples of suitable attachments means are weak adhesives, easily frangible stitching, Velcro™ fastenings, and the like. Once the diaper rash product has been applied, the applicator means and pocket 300 can be disposed of.

### Diaper Rash Product

As discussed above, the pocket is filled with a measured amount of diaper rash product. The amount of diaper rash product will generally fall between about 1 and about 50 ml per pocket. In an alternative embodiment of the present invention, the amount of diaper rash product is less than about 45 ml per pocket. In an alternative embodiment of the present invention, the amount of diaper rash product is less than about 40 ml per pocket. In an alternative embodiment of the present invention, the amount of diaper rash product is between about 1 and about 35 ml per pocket. In an alternative embodiment of the present invention, the amount of diaper rash product is between about 1 and about 30 ml per pocket. In an alternative embodiment of the present invention, the amount of diaper rash product is between about 1 and about 25 ml per pocket. In an alternative embodiment of the present invention, the amount of diaper rash product is between about 1 and about 20 ml per pocket. In an alternative embodiment of the present invention, the amount of diaper rash product is between about 1 and about 15 ml per pocket. In an alternative embodiment of the present invention, the amount of diaper rash product is between about 1 and about 10 ml per pocket. In an alternative embodiment of the present invention, the amount of diaper rash product is between about 1 and about 5 ml per pocket. In an alternative embodiment of the present invention, the amount of diaper rash product is between about 5 and about 50 ml per pocket. In an alternative embodiment of the present invention, the amount of diaper rash product is between about 5 and about 45 ml per pocket. In an alternative embodiment of the present invention, the amount of diaper rash product is between about 5 and about 40 ml per pocket. In an alternative embodiment of the present invention, the amount of diaper rash product is between about 5 and about 35 ml per pocket. In an alternative embodiment of the present invention, the amount of diaper rash product is between about 5 and about 30 ml per pocket. In an alternative embodiment of the present invention, the amount of diaper rash product is between about 5 and about 25 ml per pocket. In an alternative embodiment of the present invention, the amount of diaper rash product is between about 5 and about 20 ml per pocket. In an alternative embodiment of the present invention, the amount of diaper rash product is between about 5 and about 15 ml per pocket. In an alternative embodiment of the present invention, the amount of diaper rash product is between about 5 and about 10 ml per pocket. In an alternative embodiment of the present invention, the amount of diaper rash product is between about 10 and about 50 ml per pocket. In an alternative embodiment of the present invention, the amount of diaper rash product is between about 10 and about 45 ml per pocket. In an alternative embodiment of the present invention, the amount of diaper rash product is between about 10 and about 40 ml per pocket. In an alternative embodiment of the present invention, the amount of diaper rash product is between about 10 and about 35 ml per pocket. In an alternative embodiment of the present invention, the amount of diaper rash product is between about 10 and about 30 ml per pocket. In an alternative embodiment of the present invention, the amount of diaper rash product is between about 10 and about 25 ml per pocket. In an alternative embodiment of the present invention, the amount of diaper rash product is between about 10 and about 20 ml per pocket. In an alternative embodiment of the present invention, the amount of diaper rash product is between about 10 and about 15 ml per pocket. In an alternative embodiment of the present invention, the amount of diaper rash product is between about 15 and about 50 ml per pocket. In an alternative embodiment of the present invention, the amount of diaper rash product is between about 15 and about 45 ml per pocket. In an alternative embodiment of the present invention, the amount of diaper rash product is between about 15 and about 40 ml per pocket. In an alternative embodiment of the present invention, the amount of diaper rash product is between about 15 and about 35 ml per pocket. In an alternative embodiment of the present invention, the amount of diaper rash product is between about 15 and about 30 ml per pocket. In an alternative embodiment of the present invention, the amount of diaper rash product is between about 15 and about 25 ml per pocket. In an alternative embodiment of the present invention, the amount of diaper rash product is between about 15 and about 20 ml per pocket. In an alternative embodiment of the present invention, the amount of diaper rash product is between about 20 and about 50 ml per pocket. In an alternative embodiment of the present invention, the amount of diaper rash product is between about 20 and about 45 ml per pocket. In an alternative embodiment of the present invention, the amount of diaper rash product is between about 20 and about 40 ml per pocket. In an alternative embodiment of the present invention, the amount of diaper rash product is between about 20 and about 35 ml per pocket. In an alternative embodiment of the present invention, the amount of diaper rash product is between about 20 and about 30 ml per pocket. In an alternative embodiment of the present invention, the amount of diaper rash product is between about 20 and about 25 ml per pocket. In an alternative embodiment of the present invention, the amount of diaper rash product is between about 25 and about 50 ml per pocket. In an alternative embodiment of the present invention, the amount of diaper rash product is between about 25 ml and about 45 ml per pocket. In an alternative embodiment of the present invention, the amount of diaper rash product is between about 25 ml and about 40 ml per pocket. In an alternative embodiment of the present invention, the amount of diaper rash product is between about 25 ml and about 35 ml per pocket. In an alternative embodiment of the present invention, the amount of diaper rash product is between about 25 ml and about 30 ml per pocket. In an alternative embodiment of the present invention, the amount of diaper rash product is between about 30 ml and about 50 ml per pocket. In an alternative embodiment of the present invention, the amount of diaper rash product is between about 30 and about 45 ml per pocket. In an alternative embodiment of the present invention, the amount of diaper rash product is between about 30 and about 40 ml per pocket. In an alternative embodiment of the present invention, the amount of diaper rash product is between about 30 ml and about 35 ml per pocket. In an alternative embodiment of the present invention, the amount of diaper rash product is between about 35 ml and about 50 ml per pocket. In an alternative embodiment of the present invention, the amount of diaper rash product is between about 35 ml and about 45 ml per pocket. In an alternative embodiment of the present invention, the amount of diaper rash product is between about 35 and about 40 ml per pocket. In an alternative embodiment of the present invention, the amount of diaper rash product is between about 40 and about 50 ml per pocket. In an alternative embodiment of the present invention, the amount of diaper rash product is between about 40 and about 45 ml per pocket. However, a worker skilled in the art will appreciate that the necessary amount of diaper rash product required to treat a single infant is dependant on various factors such as the age and size of the infant, the viscosity of the particular diaper rash product being used and the necessary coverage in order to create an adequate protective barrier and can adjust the amount of diaper rash product accordingly. The actual formulation of the diaper rash product is not critical to the present invention and various different formulations of diaper rash product known in the art and can be utilised without affecting the function of the present invention. See, for example U.S. Pub. No. 20060073218 to D'Amelio et al.; U.S. Pat. No. 6,855,326 to Palumbo, et al.; U.S. Pat. No. 3,964,486 to Blaney; U.S. Pat. No. 5,618,529 to Pichierri; U.S. Pat. No. 5,091,193 to Enjolras, et al.; and U.S. Pat. No. 5,762,945 to Ashley. A worker skilled in the art would appreciate that different formulations of diaper rash product containing additional therapeutic ingredients, such as vitamins, moisturizing agents, aloe extract, antibacterial agents and the like, may be desirable due to ancillary benefits aside from the primary moisture barrier function such as promoting skin healing and wellness. Such diaper rash products which are well known in the art and are commercially available are also suitable for use with the system of the present invention.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

Although the invention has been described with reference to certain specific embodiments, various modifications thereof will be apparent to those skilled in the art without departing from the spirit and scope of the invention as outlined in the claims appended hereto.

1. A system for delivery of a diaper rash product to an affected area of skin, said system comprising:
one or more pockets formed of an upper layer and a lower layer, wherein said upper layer overlays said lower layer, and the peripheral edge of the upper layer is sealingly affixed to the lower layer to form a sealed enclosure;
each of said one or more pockets containing diaper rash product; and
a dispensing means associated with each of said one or more pockets for dispensing said diaper rash product.
wherein said one or more pockets are attached to a surface of a disposable diaper.

2. The system of claim 1, wherein said surface comprises an inner surface and/or an outer surface.

3. (canceled)

4. The system of any one of claim 1, wherein said one or more pockets are removably attached to the surface of said disposable diaper and wherein said lower layer comprises a pull tab, which extends from a peripheral edge of said lower layer but which is not attached to said surface of said disposable diaper; said pull tab for separating said lower layer away from said surface of said disposable diaper.

5. The system of claim 2, wherein the lower layer of said one or more pockets is formed of the inner surface of said disposable diaper.

6. The system of claim 1, wherein the amount of the diaper rash product is between about 1 mL and about 50 mL.

7. (canceled)

8. The system of claim 1, wherein the dispensing means comprises a pull tab, which extends from a peripheral edge of said upper layer but which is not sealed to said lower layer; said pull tab for separating said upper layer away from said lower layer.

9. The system of claim 1, wherein the dispensing means comprises a frangible seal comprising a scored line in the outer surface of said upper layer, wherein pressure on said upper layer on either side of said frangible seal causes said upper layer to break along said scored line.

10. The system of claim 1, wherein the dispensing means comprises:
a slit in said upper layer and
a strip removably attached to said upper layer, said strip covering and sealing the edges of said slit, and comprising a pull tab that is not attached to said upper layer; for removing said strip from said upper layer.

11. A method for delivering a diaper rash product to an area of skin, said method comprising the steps of:
providing a sealed pocket containing said diaper rash product, said sealed pocket operatively associated with a dispensing means; wherein said pocket is attached to a surface of a disposable diaper;
dispensing said diaper rash product through said dispensing means to provide dispensed diaper rash product that remains associated with said pocket;
applying said dispensed diaper rash product from said pocket to said area of skin.

12. The method of claim 11, wherein said surface comprises an inner surface and/or an outer surface.

13. (canceled)

14. The method of claim 11, wherein the lower layer of said one or more pockets is formed of the inner surface of said disposable diaper.

15. The method of claim 11, wherein the amount of the diaper rash product is between about 1 mL and about 50 mL.

16. (canceled)

17. The method of claim 11, wherein the dispensing means comprises a pull tab, which extends from a peripheral edge of said upper layer but which is not sealed to said lower layer; said pull tab for separating said upper layer away from said lower layer.

18. The method of claim 11, wherein the dispensing means comprises a frangible seal comprising a scored line in the outer surface of said upper layer, wherein pressure on said upper layer on either side of said frangible seal causes said upper layer to break along said scored line.

19. The method of claim 11, wherein the dispensing means comprises:
a slit in said upper layer and
a strip removably attached to said upper layer, said strip covering and sealing the edges of said slit, and comprising a pull tab that is not attached to said upper layer; for removing said strip from said upper layer.

20. A system for delivery of a diaper rash product to an affected area of skin, said system comprising:
one or more pockets formed of an upper layer and a lower layer, wherein said upper layer overlays said lower layer, and the peripheral edge of the upper layer is sealingly affixed to the lower layer to form a sealed enclosure;
each of said one or more pockets containing a measured amount of diaper rash product;
a dispensing means associated with each of said one or more pockets for dispensing said diaper rash product; and
an applicator means attached to a surface of a disposable diaper and operatively associated with one or more of said one or more pockets, wherein said applicator means comprises a tube comprising an open end and a sealed end, said sealed end being attached to said lower layer of said pocket.

21. The system of claim 20, wherein said surface comprises an inner surface and/or an outer surface of the disposable diaper.

22. (canceled)

23. The system of claim 20 wherein said one or more pockets are removably attached to the surface of said disposable diaper.

24. The system of claim 20 wherein said tube is formed of the inner surface of said disposable diaper.

25. The system of claim 20, wherein the amount of the diaper rash product is between about 1 mL and about 50 mL.

26. (canceled)

27. The system claim 20, wherein the dispensing means comprises a pull tab, which extends from a peripheral edge of said upper layer but which is not sealed to said lower layer; said pull tab for separating said upper layer away from said lower layer.

28. The system claim 20, wherein the dispensing means comprises a frangible seal comprising a scored line in the outer surface of said upper layer, wherein pressure on said upper layer on either side of said frangible seal causes said upper layer to break along said scored line.

29. The system claim 20, wherein the dispensing means comprises:
a slit in said upper layer and
a strip removably attached to said upper layer, said strip covering and sealing the edges of said slit, and comprising a pull tab that is not attached to said upper layer; for removing said strip from said upper layer.
